(19) United States
(12) Patent Application Publication
(10) Pub. No.: US 2022/0273313 A1
(43) Pub. Date: Sep. 1, 2022

(54) GASTROESOPHAGEAL AORTIC OCCLUSION DEVICE AND METHOD

(71) Applicant: THE REGENTS OF THE UNIVERSITY OF MICHIGAN, Ann Arbor, MI (US)

(72) Inventors: Kevin Ward, Glen Allen, VA (US); Jeffrey Stephen Plott, Algonac, MI (US)

(21) Appl. No.: 17/632,114

(22) PCT Filed: Aug. 18, 2020
(86) PCT No.: PCT/US20/46796

§ 371 Date: Feb. 1, 2022

Related U.S. Application Data
(60) Provisional application No. 62/888,602, filed on Aug. 19, 2019.

(51) Int. Cl. A61B 17/12; A61M 25/00; A61M 25/10

(52) U.S. Cl. CPC A61B 17/12136; A61B 17/12099; A61M 25/008; A61M 25/1002; A61M 2025/0002

(57) ABSTRACT

A device and method for occluding the descending aorta includes inserting a gastroesophageal resuscitative aortic occlusion device into a stomach of a patient through the esophagus. The gastroesophageal resuscitative aortic occlusion device includes a catheter having a body and a first lumen, and a distal end having a first opening fluidly coupled to the first lumen. An inflatable balloon is disposed on the catheter. An interior of the inflatable balloon is fluidly coupled to the first opening. An inflation device is operably connected to the catheter and fluidly coupled to the first lumen. Activation of the inflation device forces fluid into the interior of the inflatable balloon through the first lumen and the first opening. Activating the inflation device to pressurizes the inflatable balloon with a fluid. An external pressure device applies pressure to the abdomen of the patient until blood flow through the descending aorta is reduced or stopped.

### FIELD OF THE DISCLOSURE

The present disclosure relates to arterial hemorrhage occlusion devices and methods and more particularly to gastroesophageal aortic occlusion devices and methods.

### BACKGROUND

Hemorrhagic shock is a leading cause of death from trauma. Many times there are delays in reaching hospitals which are qualified to take care of the complex injuries of such individuals. Many patients who die of trauma, die from multi-system involvement. Multi-system involvement may include head injury along with injuries to organs of the thoracic and abdominal cavity. Uncontrolled hemorrhage leading to hypovolemic shock is a leading cause of death from trauma especially from blunt and penetrating trauma of the abdomen. When head trauma occurs concomitantly with thoracic and abdominal hemorrhage, the brain becomes hypoperfused and, thus, becomes at greater risk for secondary injury. Currently, in the pre-hospital and emergency department setting, there are limited means to control exsanguinating hemorrhage below the diaphragm while maintaining myocardial and cerebral blood flow. Definitive control of hemorrhage is performed at surgery but this may be delayed and may not occur within the golden hour (time from injury to definitive treatment/repair) where the best opportunity lies in salvaging the patient. Survival with improved neurologic outcome might be enhanced if means were available to slow or stop ongoing hemorrhage (especially below the diaphragm) while maintaining adequate perfusion to the heart and brain until definitive treatment of the hemorrhage is available. This would be especially true of trauma victims whose transport to appropriate medical facilities would be prolonged.

One method of slowing or stopping hemorrhage is the use of a pneumatic anti-shock garment (PASG). Use of the PASG has met with varying degrees of success depending on the location of injury. This garment is placed on the legs and abdomen and is then inflated. Hemorrhage in the abdominal cavity, as well as the lower extremities, is controlled through tamponade while systemic blood pressure is raised partially through autotransfusion and by raising peripheral vascular resistance. Use of the PASG can sometimes be cumbersome and does not uniformly control hemorrhage or raise blood pressure. In addition, persons with concomitant penetrating thoracic injuries may hemorrhage more when the device is applied. The device may also raise intracranial pressure, which might detrimentally alter cerebral blood flow resulting in neurologic injury.

Other more drastic means to control abdominal bleeding prior to surgery have been the use of thoracotomy to cross-clamp the thoracic aorta and the use of balloon catheters placed into the aorta from the femoral arteries to a point above the celiac-aortic axis. These techniques have met with varying degrees of success and require a high degree of skill and cannot be performed in hospitals not equipped to care for trauma patients or by paramedical care personnel.

Deliberately keeping hemorrhaging trauma victims in a hypotensive state is currently being examined as a means to improve survival. This is done based on the premise that overall hemorrhage (especially abdominal hemorrhage) is reduced if mean arterial pressure is kept low by not aggressively volume-repleting the victim prior to surgery. Unfortunately, this may be dangerous for trauma victims with concomitant head injury or myocardial dysfunction.

An important cause of hemorrhagic shock not caused by trauma includes rupture of abdominal aortic aneurysms. These can occur suddenly and without warning. Control of bleeding even at surgery can be difficult. Temporary measures discussed above for hemorrhage secondary to trauma have been tried for hemorrhage secondary to aneurysm rupture. The same difficulties apply. Survival might be enhanced if hemorrhage could be controlled earlier while maintaining perfusion to the heart and brain.

U.S. Pat. No. 5,531,776, and U.S. Patent Publication No. 2002/0016608, the disclosures of which are hereby incorporated herein by reference, each disclose non-invasive techniques for partially or completely occluding the descending aorta. While these techniques have been at least somewhat successful at reducing hemorrhagic shock, these methods and devices have not gained widespread acceptance due to some difficulty in advancing and properly placing the devices in a patient. Additionally, these methods require particular orientations of the devices for correct operation. Finding and maintaining the precise orientation can be difficult.

### SUMMARY

In a first example, a gastroesophageal resuscitative aortic occlusion device includes a catheter, the catheter including a body having a first lumen, the body also having a distal end including a first opening fluidly coupled to the first lumen. An inflatable balloon is disposed on the catheter. An interior of the selectively inflatable balloon is fluidly coupled to the first opening. An inflation device is operably connected to the catheter and fluidly coupled to the first lumen. Activation of the inflation device forces fluid into the interior of the inflatable balloon through the first lumen and the first opening.

In a second example, a gastroesophageal resuscitative aortic occlusion kit includes an occlusion device having a catheter, the catheter including a body having a first lumen. The body has a distal end including a first opening fluidly coupled to the first lumen. An inflatable balloon is disposed on the catheter. An interior of the selectively inflatable balloon is fluidly coupled to the first opening. An inflation device is operably connected to the catheter and fluidly coupled to the first lumen. Activation of the inflation device forces fluid into the interior of the inflatable balloon through the first lumen and the first opening. An external pressure device is used in conjunction with the occlusion device.

In yet another example, a method of occluding the descending aorta includes inserting a gastroesophageal resuscitative aortic occlusion device into a stomach of a patient through the esophagus. The gastroesophageal resuscitative aortic occlusion device includes a catheter having a body and a first lumen. The body also includes a distal end having a first opening fluidly coupled to the first lumen. An inflatable balloon is disposed on the catheter. An interior of the inflatable balloon is fluidly coupled to the first opening. An inflation device is operably connected to the catheter and fluidly coupled to the first lumen. Activation of the inflation device forces fluid into the interior of the inflatable balloon through the first lumen and the first opening. Activating the inflation device to pressurizes the inflatable balloon with a fluid. An external pressure device applies pressure to the abdomen of the patient until blood flow through the descending aorta is reduced or stopped.

Any of the first, second, and third examples may include any one or more of the following optional forms.

In one optional form, a first vacuum device is operably connected to the catheter, the first vacuum device activating to remove fluid from the inflatable balloon.

In another optional form, the first vacuum device is fluidly coupled to the first lumen.

In yet other optional forms, the body further includes a second lumen and the distal end further includes a second opening fluidly coupled to the second lumen, the second lumen fluidly connecting the distal end to ambient pressure.

In yet other optional forms, the body further includes a third lumen and the distal end further includes a third opening fluidly coupled to the third lumen.

In yet other optional forms, a second vacuum device is fluidly coupled to the third lumen, the second vacuum device activating to remove stomach contents when the distal end is located in a patient stomach.

In yet other optional forms, the inflatable balloon may be displaced from the distal end of the catheter by a distance, preferably by at least 20 mm.

In yet other optional forms, the inflatable balloon is displaced from the distal end of the catheter by between about 20 mm and about 150 mm, preferably between about 30 mm and about 100 mm.

In yet other optional forms, the inflatable balloon comprises a material having a shore hardness of between 70A and 70D.

In yet other optional forms, the inflatable balloon further comprises a wall having a thickness of between 0.003 in and 0.015 in.

In yet other optional forms, a pressure sensor senses internal pressure in the inflatable balloon.

In yet other optional forms, a pulsatile flow sensor senses blood pressure.

In yet other optional forms, the fluid is ambient air.

In yet other optional forms, operation of the inflation device may be reversed to remove fluid from the inflatable balloon.

In yet other optional forms, the distal end of the catheter comprises a plurality of cutouts to increase flexibility.

In yet other optional forms, the distal end of the catheter comprises a softer material than the catheter proximate the inflatable balloon.

In yet other embodiments, the distal end of the catheter comprises a smaller diameter than the catheter proximate the inflatable balloon.

In yet other optional forms, the distal end of the catheter comprises a blunt end.

In yet other optional forms, a removable sheath covers the inflatable balloon during insertion of the catheter.

In yet other optional forms, the inflatable balloon comprises an annular shape when fully inflated.

In yet other optional forms, an external pressure device is applied to the abdomen of the patient.

In yet other optional forms, the external compression device is one of a circumferential compression device and a human hand.

In yet other optional forms, a telescoping member may be at least partially slidably disposed within the catheter and the catheter may slide along the telescoping member for insertion, removal, and/or positioning within a patient.

### BRIEF DESCRIPTION OF THE DRAWINGS

FIG. 1 is a schematic view of a gastroesophageal resuscitative aortic occlusion device inserted into a stomach of a patient.

FIG. 2 is a plan view of the gastroesophageal resuscitative aortic occlusion device of FIG. 1

FIG. 3 is a close up view of a balloon of the gastroesophageal resuscitative aortic occlusion device of FIG. 2 in a collapsed state.

FIG. 4A is a close up view of a distal end of a catheter of the gastroesophageal resuscitative aortic occlusion device of FIG. 2.

FIG. 4B is a cross sectional view of the catheter of FIG. 4A

FIG. 5 is a perspective view of an external pressure device applied to an abdomen of a patient and used in conjunction with the gastroesophageal resuscitative aortic occlusion device of FIG. 2.

FIG. 6 is a plan view of the external pressure device of FIG. 5.

FIGS. 7A-7C are side perspective, close up perspective, and cross-sectional views, respectively, of an alternate embodiment of a gastroesophageal resuscitative aortic occlusion device, with a telescoping member at least partially deployed.

FIGS. 8A-8C are side perspective, close up perspective, and cross-sectional views, respectively, of the gastroesophageal resuscitative aortic occlusion device of FIGS. 7A-7C, with the telescoping member retracted.

FIGS. 9A and 9B are side and close up views, respectively, of another alternate embodiment of a gastroesophageal resuscitative aortic occlusion device, with a telescoping member at least partially deployed.

FIGS. 10A and 10 B are close-up and partial cross-sectional views, respectively of the gastroesophageal resuscitative aortic occlusion device of FIGS. 9A and 9B, with the telescoping member retracted.

### DETAILED DESCRIPTION

The disclosed devices and techniques are based upon the fact that a majority of human beings have similar physiological relationships between the esophagus, the stomach, and the descending aorta. These methods include positioning a device, having an elongated tubular member, in a portion of the patient's stomach juxtaposed with the patient's descending aorta and displacing with the tubular member a wall of the portion of the stomach posteriorly-laterally in the direction of the descending aorta.

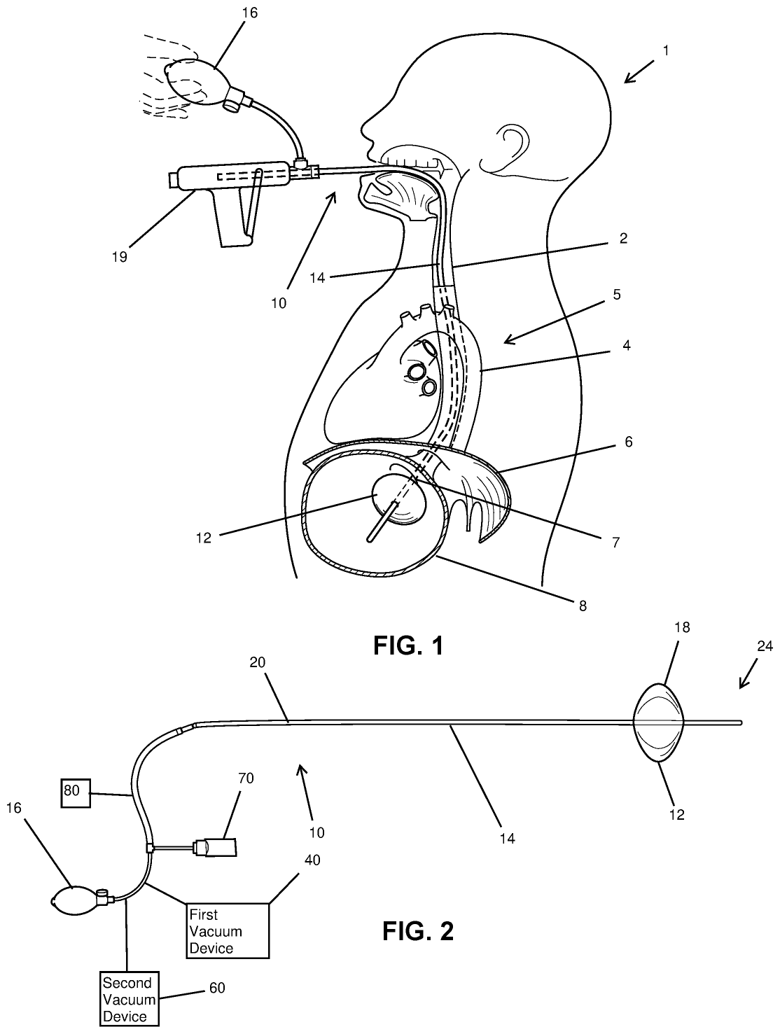

Turning now to FIG. 1, one embodiment of a gastroesophageal resuscitative aortic occlusion device 10 is illustrated inserted into a patient 1 through the patient's esophagus 2. The patient's descending aorta 4 is juxtaposed with the esophagus 2 throughout a significant portion of the thoracic cavity 5. However, the esophagus 2 and descending aorta 4 are most closely bound where they mutually pass in close proximity through the diaphragm 6 just above the esophageal-gastric junction 7. Below the diaphragm 6, the descending aorta 4 passes posteriorly of the stomach 8 between the stomach 8 and vertebral spinal column. Because the descending aorta 4 and esophagus 2 are tightly bound in close proximity where they pass through the diaphragm 6, manipulation of a device positioned adjacent the esophageal-gastric junction 7 may be used to deflect or expand the esophagus 2 and/or the stomach 8 to thereby at least partially occlude the descending aorta 4 against the vertebral column, which decreases or stops blood flow through the descending aorta 4.

To carry out such non-invasive partial or complete occlusion of the descending aorta 4 requires proper positioning both longitudinally and radially of a surface which is moveable laterally a sufficient distance, with a sufficient force, and having a surface of sufficient area to at least partially occlude the patient's descending aorta 4. The gastroesophageal resuscitative aortic occlusion device 10 described herein overcomes the difficulties of proper positioning of the moveable surface notwithstanding the great variety in the anatomy of a patient, as will be described in more detail below.

The gastroesophageal resuscitative aortic occlusion device 10 includes a force-producing surface, such as an inflatable balloon 12, and a positioning device in the form of an elongated member, such as a catheter 14. The catheter 14 positions the inflatable balloon 12 through the patient's esophagus 2 and into a portion of the patient's stomach 8, which is near the patient's descending aorta 4. The gastroesophageal resuscitative aortic occlusion device 10 further includes an inflation mechanism, such as a hand pump 16 which selectively inflates the inflatable balloon 12. The outer surface 18 of the inflatable balloon 12 applies pressure posteriorly-laterally in the direction of the patient's descending aorta 4 sufficient to cause either partial, or substantially complete, occlusion of the patient's descending aorta 4.

Once inflated in the patient's stomach 8, proper positioning of the inflatable balloon 12 is achieved by pulling the catheter 14, such as by pulling on a handgrip 19, which draws the inflatable balloon 12 to the wall of the stomach 8 at the esophageal-gastric junction 7. The inflatable balloon 12 is drawn upwardly and posteriorly, which is the direction necessary to impinge the descending aorta 4, thereby substantially occluding blood flow through the patient's descending aorta 4.

The descending aorta 4 is a main artery of the body. As such, it is a large vessel and it is pressurized by the heart to a pressure that may extend over 200 millimeters of mercury, or approximately four pounds per square inch in some cases. Therefore, in order to substantially occlude the descending aorta 4, the gastroesophageal resuscitative aortic occlusion device 10 must overcome pressures as great as 200 mm of mercury. Furthermore, the descending aorta 4 is a muscular structure having muscle tone which affords rigidity. Therefore, the descending aorta 4 has a stiffness which resists crushing thereof. While less than the pressure of the fluid in the descending aorta 4, this muscle tone adds appreciably to the force required to substantially occlude the descending aorta 4. In the illustrated embodiment, force sufficient to partially or completely occlude the descending aorta 4 is achieved with the inflatable balloon 12 being made of a suitable medical grade material, such as polyurethane, a polyester film, such as Mylar®, polyetheylene terephthalate, or similar materials, and having a surface with an inflated diameter preferably of between approximately 3 and approximately 8 inches and most preferably between approximately 5 and approximately 7 inches. Internal balloon pressures of between 30 mm of mercury and 500 mm of mercury, preferably between 60 mm of mercury and 300 mm of mercury, advantageously allow the inflatable balloon 12 to partially or fully occlude the descending aorta and/or to reduce or stop bleeding in other areas of the abdomen. The catheter 14 is sufficiently strong to allow forces to be transmitted to inflatable balloon 12 to impart force to the surface of inflatable balloon 12 to partially or completely occlude the descending aorta 4.

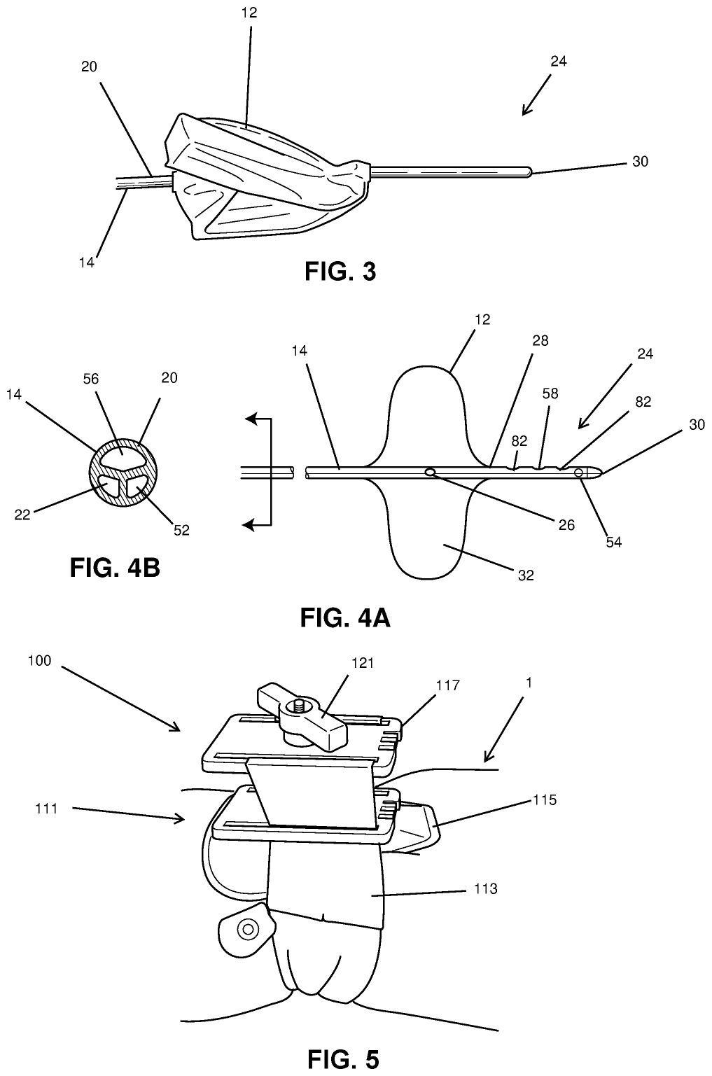

As illustrated in FIGS. 2-4, the gastroesophageal resuscitative aortic occlusion device 10 comprises the catheter 14, which has a body 20 including a first lumen 22. The body 20 also includes a distal end 24 having a first opening 26 fluidly coupled to the first lumen 22.

The inflatable balloon 12 is disposed on the catheter 14 and a distal end 28 of the inflatable balloon 12 is displaced from a tip 30 of the distal end 24 of the catheter 14 by a distance. An interior 32 of the inflatable balloon 12 is fluidly coupled to the first opening 26.

In some embodiments, the distal end 28 of the inflatable balloon 12 is displaced from the tip 30 of the distal end 24 of the catheter 14 by at least 20 mm. In other embodiments, the distal end 28 of the inflatable balloon 12 is displaced from the tip 30 of the distal end 24 of the catheter 14 by between about 20 mm and about 150 mm, preferably by between about 30 mm and about 100 mm. Displacements in these ranges facilitate insertion and proper placement of the inflatable balloon 12 into the stomach 8.

In some embodiments, the inflatable balloon 12 comprises a material having a shore hardness of between 70A and 70D. In other embodiments, the inflatable balloon 12 further comprises a wall having a thickness of between 0.003 in and 0.015 in. These ranges of hardness and wall thickness produce sufficient force to occlude or partially occlude the descending aorta 4 when the inflatable balloon 12 is inflated.

The hand pump 16 is operably connected to the catheter 14 and fluidly coupled to the first lumen 22. Activation of the hand pump 16, such as by squeezing, forces fluid, such as ambient air, into the interior 32 of the inflatable balloon 12 under pressure through the first lumen 22 and through the first opening 26. The inflatable balloon 12 is thereby filled with fluid under pressure, which causes the inflatable balloon 12 to expand.

In some embodiments, an optional a first vacuum device 40 may be operably connected to the catheter 14, the first vacuum device 40 activating to remove fluid from the inflatable balloon 12, thereby causing the inflatable balloon 12 to deflate. The first vacuum device 40 may be fluidly connected to the first lumen 22. The first vacuum device 40 may be used to deflate the inflatable balloon 12 during insertion, repositioning, and/or during removal of the inflatable balloon 12 from the patient 1. In some embodiments, the hand pump 16 and the first vacuum device 40 may be combined into a single device, such as a hand pump with a reversible valve to allow fluid to be pumped or removed based on the valve position.

In some embodiments, the body 20 may further include a second lumen 52 and the distal end 24 of the catheter 14 may further include a second opening 54 that is fluidly coupled to the second lumen 52. The second lumen 52 may allow the second opening 54 at the distal end 24 to be fluidly connected with ambient pressure, which equalizes pressure in the stomach 8 of the patient 1 when the inflatable balloon 12 is inflated, which may prevent equalization of pressure through the esophagus 2.

In some embodiments, the body 20 may further include a third lumen 56 and the distal end 24 of the catheter 14 may further include a third opening 58, which may be fluidly coupled to the third lumen 56. The third lumen 56 may be optionally fluidly connected to a second vacuum device 60. The second vacuum device 60 may be activated to remove stomach contents when the distal end 24 is located in the patient's stomach 8. In some cases, stomach contents may need to be removed to make room for the inflatable balloon 12, or to increase effectiveness of the inflatable balloon 12, and/or to reduce the risk of patient aspiration.

In some embodiments, the gastroesophageal resuscitative aortic occlusion device 10 further includes a pressure sensor 70 that senses internal pressure in the inflatable balloon 12. The pressure sensor 70 may be fluidly connected to the first lumen 22 so that internal pressure of the inflatable balloon 12 may be sensed so that a user may inflate the inflatable balloon 12 to the proper pressure.

In yet other embodiments, the gastroesophageal resuscitative aortic occlusion device 10 may further include a pulsatile flow sensor or pressure sensor 80, either embedded into the device, or that are operatively connected to the device but that are applied external to the patient distal to the point of balloon inflation, that sense blood pressure or flow emanating from the descending aorta 4 distal to the point of balloon inflation. In some embodiments, the sensors may be separate from the balloon apparatus, but part of a larger system. The sensors provide data that enhances positioning of the balloon to aid in more complete occlusion of the descending aorta. In some uses, it may be desirable to only partially occlude the descending aorta, for example, if an operator determines that blood flow should only be slowed, but not stopped for medical reasons, in which case the sensors provide data that will assist in producing the desired amount of occlusion. In some embodiments, the sensors may comprise conductive bands or other pressure sensing elements in the balloon, or optical pressure sensors or ultrasonic pressure sensors for internal sensing, or external sensing (relative to the device itself) that may be attached to other bodily structures in the patient distal to the location of balloon inflation.

In some embodiments, the distal end 24 of the catheter 14 may include a plurality of cutouts 82 that increase flexibility of the distal end 24 to ease insertion of the catheter 14 through the esophagus 2.

In yet other embodiments, the distal end 24 of the catheter 14 may comprise a softer material than the catheter 14 proximate the inflatable balloon 12.

In some embodiments, the tip 30 of the distal end 24 of the catheter 14 comprises a blunt end.

In yet other embodiments, the gastroesophageal resuscitative aortic occlusion device 10 may comprise a removable sheath (not shown) that covers the inflatable balloon 12 during insertion of the inflatable balloon 12 through the esophagus 2. The sheath may break-away or dissolve when contacted with stomach acid to free the inflatable balloon 12 for inflation.

In yet other embodiments, the inflatable balloon 12 comprises an annular shape when fully inflated.

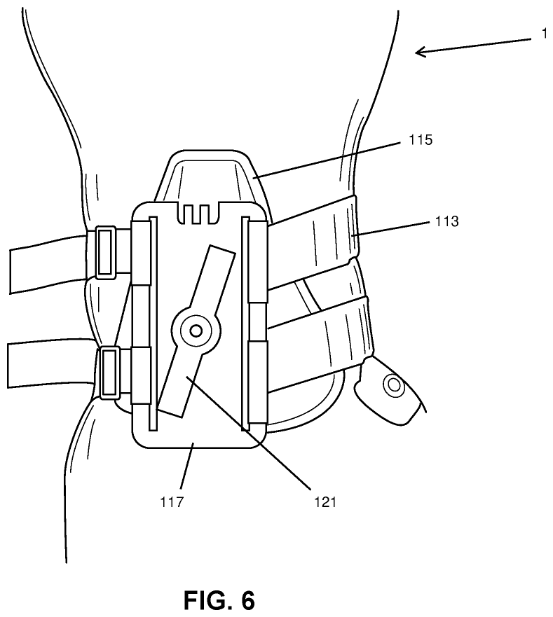

Turning now to FIGS. 5 and 6, an external pressure device 100 may be used in conjunction with the gastroesophageal resuscitative aortic occlusion device 10 to enhance occlusion of the descending aorta 4.

In some embodiments, the external pressure device 100 may comprise a circumferential compression device 111 that includes a strap 113 and a pressure plate 115. In other embodiments, the external pressure device 100 may comprise a human hand.

In the embodiment illustrated in FIGS. 5 and 6, the circumferential compression device 111 may also include an upper plate 117 that is movably connected to the pressure plate 115. The upper plate 117 may include a pressure increasing device, such as an extending screw 121 that increases pressure of the pressure plate 115 on the abdomen of the patient 1.

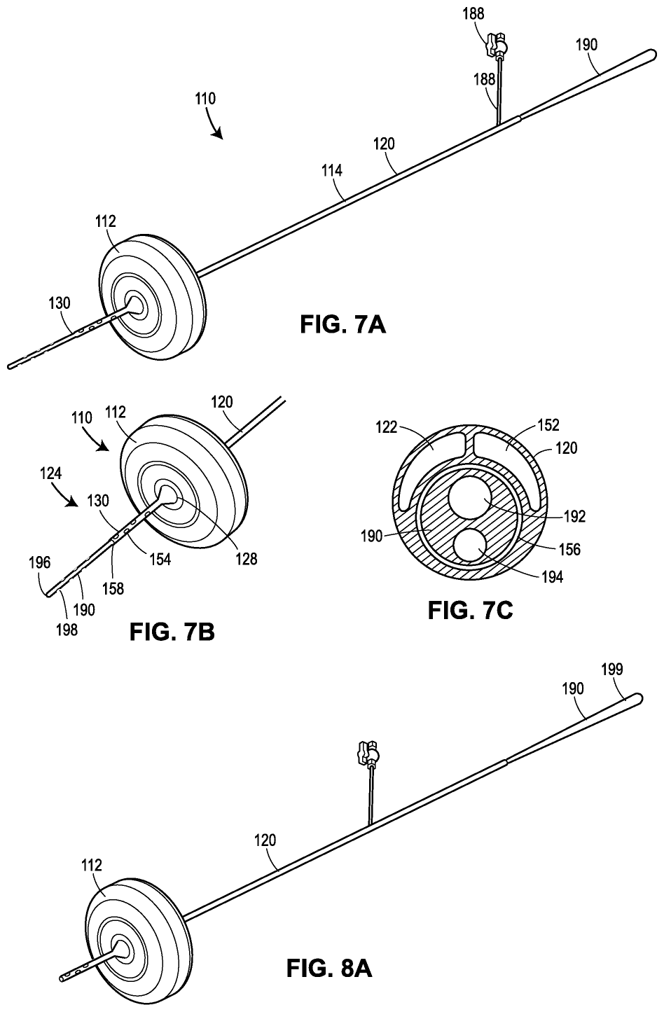

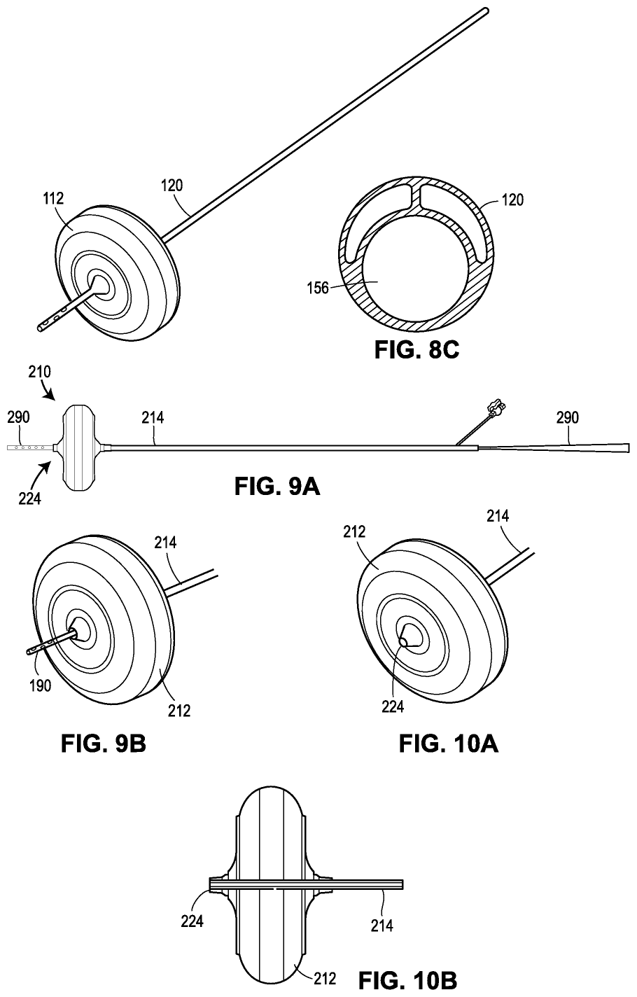

Turning now to FIGS. 7-10, other optional embodiments of a gastroesophageal resuscitative aortic occlusion device are illustrated. The gastroesophageal resuscitative aortic occlusion devices of FIGS. 7-10 may include any features described above with respect to FIGS. 1-4, even if they are not expressly described with respect to FIGS. 7-10. Moreover, the embodiment described in FIGS. 7-8 has common elements listed as 100 greater than the elements of FIGS. 1-4 and the embodiment described in FIGS. 9-10 has common elements listed as 200 greater than the elements of FIGS. 1-4.

The gastroesophageal resuscitative aortic occlusion device 110 of FIGS. 7 and 8 comprises a catheter 114, which has a body 120 including a first lumen 122. The body 120 also includes a distal end 124 having a first opening located within an inflatable balloon 112 (but not illustrated in FIGS. 7 and 8) fluidly coupled to the first lumen 122.

The inflatable balloon 112 is disposed on the catheter 114 and a distal end 128 of the inflatable balloon 112 is displaced from a tip 130 of the distal end 124 of the catheter 114 by a distance. An interior of the inflatable balloon 112 is fluidly coupled to the first opening.

In some embodiments, the distal end 128 of the inflatable balloon 112 is displaced from the tip 130 of the distal end 124 of the catheter 114 by at least 20 mm. In other embodiments, the distal end 128 of the inflatable balloon 112 is displaced from the tip 130 of the distal end 124 of the catheter 114 by between about 20 mm and about 150 mm, preferably by between about 30 mm and about 100 mm. Displacements in these ranges facilitate insertion and proper placement of the inflatable balloon 112 into the stomach.

A hand pump (not shown in FIGS. 7 and 8) is operably connected to the catheter 114 and fluidly coupled to the first lumen 122 through a fluid line 186 and valve 188. Activation of the hand pump, such as by squeezing, forces fluid, such as ambient air, into the interior of the inflatable balloon 112 under pressure through the first lumen 122 and through the first opening. The inflatable balloon 112 is thereby filled with fluid under pressure, which causes the inflatable balloon 112 to expand.

In some embodiments, an optional a first vacuum device (not shown in FIGS. 7 and 8) may be operably connected to the catheter 114, also through the fluid line 186 and the valve 188. The first vacuum device may activate to remove fluid from the inflatable balloon 112, thereby causing the inflatable balloon 112 to deflate. The first vacuum device may also be fluidly connected to the first lumen 122. The first vacuum device may be used to deflate the inflatable balloon 112 during insertion, repositioning, and/or during removal of the inflatable balloon 112 from the patient. In some embodiments, the hand pump and the first vacuum device may be combined into a single device, such as a hand pump with a reversible valve to allow fluid to be pumped or removed based on the valve position.

In some embodiments, the body 120 may further include a second lumen 152 and the distal end 124 of the catheter 14 may further include a second opening(s) 154 that is fluidly coupled to the second lumen 152. The second lumen 152 may allow the second opening 154 at the distal end 124 to be fluidly connected with ambient pressure, which equalizes pressure in the stomach of the patient when the inflatable balloon 112 is inflated, which may prevent equalization of pressure through the esophagus.

In some embodiments, the body 120 may further include a third lumen 156 and the distal end 124 of the catheter 114 may further include a third opening 158, which may be coupled to the third lumen 156. The third lumen 156 may be sized and shaped to receive a telescoping member 190. The telescoping member 190 is slidably disposed within the third lumen 156 so that a distal end of the telescoping member 190 may extend out of the distal end 124 of the catheter 114, when the telescoping member 190 is in a deployed position.

The telescoping member 190 may facilitate placement of the inflatable balloon 112. For example, the telescoping member 190 may protrude beyond the distal end 124 of the catheter 114. There is no limitation on how far the telescoping member 190 might protrude. In one embodiment, a clinician may first place the telescoping member 190 into the patient's stomach and then pass the gastroesophageal resuscitative aortic occlusion device 110 over the telescoping member 190. Alternatively, the telescoping member 190 may only protrude partially out of the distal end 124 of the catheter 114 to provide additional guidance and support for the gastroesophageal resuscitative aortic occlusion device 110 during insertion. As a result, the gastroesophageal resuscitative aortic occlusion device 110 is more easily navigated into the esophagus and into the stomach since the telescoping member 190 acts as a guide. The telescoping member 190 is preferably flexible. The telescoping member 190 may comprise, for example, PVC or a thermoplastic polyurethane (TPU), but other flexible materials may be used as well. The telescoping member 190 may optionally include a first lumen 192 and a second lumen 194. The first and second lumens 192,194 may be utilized, for example, for the removal of stomach contents. More specifically, the first lumen 192 may be used for vacuuming out stomach contents while the second lumen 194 may be used for venting the stomach to atmosphere. A distal end 196 of the telescoping member 190 may include one or more fenestrations 198. These fenestrations 198 fluidly connect the first and second lumens 192, 194 the outside of the telescoping member 190, for example, to the stomach of a patient and any contents therein. Further the distal end 196 is preferably rounded to minimize trauma to the patient during insertion and removal.

When the telescoping member 190 is no longer needed, the clinician may partially or completely remove the telescoping member 190 by pulling on a proximal end 199. The telescoping member 190 then slides out (partially or completely) of the third lumen 156.

Turning now to FIGS. 9 and 10, another alternate embodiment of a gastroesophageal resuscitative aortic occlusion device 210 is illustrated. The gastroesophageal resuscitative aortic occlusion device 210 of FIGS. 9 and 10 is the same as the embodiment illustrated in FIGS. 7 and 8, with the exception of the inflatable balloon 212 placement on the catheter 214. In the embodiment of FIGS. 9 and 10, the inflatable balloon 112 may be mounted near or at the distal end 224 of the catheter 214. A telescoping member 290 may also be used to assist in placement of the inflatable balloon 212. Then, once the inflatable balloon 212 is positioned within the stomach, the telescoping member 290 can be removed as described above, and the inflatable balloon 212 may be inflated. Once the inflatable balloon 212 is fully inflated, it acts to shield the stomach from the distal end 224 of the catheter 214. As a result, trauma to the stomach is reduced, which could occur from prolonged force of a more pointed object pressed up against the stomach wall.

In some embodiments, a gastroesophageal resuscitative aortic occlusion kit may include the gastroesophageal resuscitative aortic occlusion device 10 and external pressure device 100 described above.

The gastroesophageal resuscitative aortic occlusion device 10 and external pressure device 100 described above may be used in a method of occluding the descending aorta 4.

The gastroesophageal resuscitative aortic occlusion device 10 may be inserted into the stomach 8 of a patient through the esophagus 2. The hand pump 16 may be activated to pressurize the inflatable balloon 12 with a fluid. External pressure is applied to the abdomen of the patient 1 with the external pressure device 100 until blood flow through the descending aorta 4 is reduced or stopped.

The disclosed gastroesophageal resuscitative aortic occlusion device, external pressure device, and methods of using the gastroesophageal resuscitative aortic occlusion device and the external pressure device provide hemorrhage control for the management of trauma and an inhibition of blood flow below the diaphragm to enhance coronary and cerebral perfusion. Studies have shown that, although over half of the tissue beds are below the diaphragm, approximately two-thirds of bleeding that leads to hemorrhagic shock occurs below the diaphragm. Therefore, the ability to control bleeding below the diaphragm provides a significant advantage particularly in management of trauma. This is particularly useful in treating patients who have suffered abdominal injuries from knives and guns, blunt trauma from falls, explosions, motor vehicle accidents, complications due to the delivery of babies from subdiaphragmatic hemorrhaging and other vascular catastrophes below the diaphragm such as ruptured abdominal aortic aneurysms. The disclosed gastroesophageal resuscitative aortic occlusion device and external pressure device are particularly useful in battlefield applications in which it is essential to be able to rapidly control life-threatening hemorrhage in a non-invasive manner in order to avoid immediate death and complications from infections and the like until definitive repair of injuries can take place. Additionally, the ability to perform this procedure rapidly and effectively reduces the exposure of the medical personnel to battlefield injuries.

Changes and modifications in the specifically described embodiments can be carried out without departing from the principles of the invention. For example, electrodes can be applied to stomach balloons for use in cardiac pacing and defibrillation. Although balloons and cuffs may be inflated using air, other techniques involving hydraulic fluids and mechanical actuators may suggest themselves to those skilled in the art. Although inflatable devices are illustrated as spherical or annular, other shapes could be used such as cylindrical, pill-shaped, and the like. Also, the various elements of each illustrated embodiment of the invention can be combined and substituted with other of the embodiments. The embodiments are provided in order to illustrate the invention and should not be considered limiting. The described methods and devices are to be limited only by the scope of the appended claims.

1. A gastroesophageal resuscitative aortic occlusion device comprising:
a catheter, the catheter including a body having a first lumen, the body also having a distal end including a first opening fluidly coupled to the first lumen;
an inflatable balloon disposed on the catheter, an interior of the inflatable balloon being fluidly coupled to the first opening; and
an inflation device operably connected to the catheter and fluidly coupled to the first lumen,
wherein activation of the inflation device forces fluid into the interior of the inflatable balloon through the first lumen and the first opening.

2. The gastroesophageal resuscitative aortic occlusion device of claim 1, further comprising a first vacuum device operably connected to the catheter, the first vacuum device activating to remove fluid from the inflatable balloon.

3. The gastroesophageal resuscitative aortic occlusion device of claim 2, wherein the first vacuum device is fluidly coupled to the first lumen.

4. The gastroesophageal resuscitative aortic occlusion device of claim 1, the body further including a second lumen and the distal end further including a second opening fluidly coupled to the second lumen, the second lumen fluidly connecting the distal end to ambient pressure.

5. The gastroesophageal resuscitative aortic occlusion device of claim 4, the body further including a third lumen and the distal end further including a third opening fluidly coupled to the third lumen.

6. The gastroesophageal resuscitative aortic occlusion device of claim 3, further comprising a second vacuum device fluidly coupled to a second lumen, the second vacuum device activating to remove stomach contents when the distal end is located in a patient stomach.

7. (canceled)

8. The gastroesophageal resuscitative aortic occlusion device of claim 1, wherein the inflatable balloon is displaced from the distal end of the catheter by between about 20 mm and about 150 mm, preferably between about 30 mm and about 100 mm.

9. (canceled)

10. The gastroesophageal resuscitative aortic occlusion device of claim 1, wherein the inflatable balloon further comprises a wall having a thickness of between 0.003 in and 0.015 in.

11. The gastroesophageal resuscitative aortic occlusion device of claim 1, further comprising a pressure sensor that senses internal pressure in the inflatable balloon.

12. The gastroesophageal resuscitative aortic occlusion device of claim 1, further comprising a pulsatile flow sensor.

13. (canceled)

14. The gastroesophageal resuscitative aortic occlusion device of claim 1, wherein operation of the inflation device may be reversed to remove fluid from the inflatable balloon.

15. The gastroesophageal resuscitative aortic occlusion device of claim 1, wherein the distal end of the catheter comprises a plurality of cutouts to increase flexibility.

16. The gastroesophageal resuscitative aortic occlusion device of claim 1, wherein the distal end of the catheter comprises one of a softer material than the catheter proximate the inflatable balloon or the distal end of the catheter comprises a smaller diameter than the catheter proximate the inflatable balloon.

17. The gastroesophageal resuscitative aortic occlusion device of claim 1, wherein the distal end of the catheter comprises a blunt end.

18. The gastroesophageal resuscitative aortic occlusion device of claim 1, further comprising a removable sheath that covers the inflatable balloon.

19. The gastroesophageal resuscitative aortic occlusion device of claim 1, wherein the inflatable balloon comprises an annular shape when fully inflated.

20. The gastroesophageal resuscitative aortic occlusion device of claim 1, further comprising an external pressure device.

21. (canceled)

22. The gastroesophageal resuscitative aortic occlusion device of claim 1, further comprising a telescoping member that is at least partially slidably disposed within the catheter.

23. (canceled)

24. A gastroesophageal resuscitative aortic occlusion kit, comprising:
an occlusion device comprising a catheter, the catheter including a body having a first lumen, the body also having a distal end including a first opening fluidly coupled to the first lumen; an inflatable balloon disposed on the catheter, an interior of the inflatable balloon being fluidly coupled to the first opening; and an inflation device operably connected to the catheter and fluidly coupled to the first lumen, wherein activation of the inflation device forces fluid into the interior of the inflatable balloon through the first lumen and the first opening; and
an external pressure device.

25. A method of occluding the descending aorta, the method comprising:
inserting a gastroesophageal resuscitative aortic occlusion device into a stomach of a patient through the esophagus, the gastroesophageal resuscitative aortic occlusion device comprising a catheter, the catheter including a body having a first lumen, the body also having a distal end including a first opening fluidly coupled to the first lumen; an inflatable balloon disposed on the catheter, an interior of the inflatable balloon being fluidly coupled to the first opening; and an inflation device operably connected to the catheter and fluidly coupled to the first lumen, wherein activation of the inflation device forces fluid into the interior of the inflatable balloon through the first lumen and the first opening;
activating the inflation device to pressurize the inflatable balloon with a fluid; and
applying external pressure to the abdomen of the patient until blood flow through the descending aorta is reduced or stopped.

26. (canceled)

27. (canceled)
